(19) United States
(12) Patent Application Publication
(10) Pub. No.: US 2012/0101421 A1
(43) Pub. Date: Apr. 26, 2012

(54) DYNAMIC SHOULDER JOINT ORTHESIS, IN PARTICULAR A SHOULDER ABDUCTION ORTHESIS, COMPRISING A FLOATINGLY MOUNTED UPPER ARM SPLINT PART

(75) Inventor: Erich ALBRECHT, Neubeuern (DE)

(73) Assignee: ALBRETCH GmbH, Neubeuern (DE)

(21) Appl. No.: 13/275,699

(22) Filed: Oct. 18, 2011

(30) Foreign Application Priority Data
Oct. 21, 2010 (DE) 102010049191.8-51

(51) Int. Cl. A61F 5/00

(52) U.S. Cl. 602/20

(57) ABSTRACT

In a shoulder joint orthesis a distal upper arm splint part (31) is mounted floatingly on a proximal upper arm splint part (30) so that the distance between an upper arm support (13) and a hinge can be changed during a movement in the adduction and abduction directions.

The invention relates to a dynamic shoulder joint orthesis, in particular a shoulder abduction orthesis, according to the preamble of claim 1.

The shoulder joint of an adult human is at risk, in a rather specific manner, of becoming immobile, particularly in terms of abduction, if it does not experience sufficient movement as a result of capsular retraction, particularly in the region of the recessus axillaris, and sticking of the displacement structures. Immobilisation damage to an otherwise healthy shoulder can only be measured after a rest period of approximately one week. Elderly patients are particularly at risk. After a surgical procedure in the case of a lesion of the capsular ligament tissue and gliding structures, the risk of development of a contracture of the shoulder joint is disproportionately higher. However, sufficient movement is extremely important for the function of the shoulder joint in order to avoid contractures. It promotes effusion and oedema resorption and helps to avoid thromboses by accelerating the blood flow.

In a relatively large number of patients, it was determined either during or after a surgical procedure owing to shoulder damage or trauma that there is a considerable restriction of the stability of the structures responsible for abduction of the upper arm. For example, this is the case after a refixing or reconstruction of the tendon of the musculus supraspinatus by sewing, then reattachment to the tuberculum majus, reconstruction by a latissimus dorsi transfer or refixing of the tuberculum majus or with fractures of the proximal humerus, wherein, when treated by means of plates, intermedullary pins or endoprosthesis, the tubercula had to be refixed to the appending tendons of the rotator cuff.

During aftercare, it is generally necessary to protect the reconstructed structures against a renewed rupture or re-dislocation for a relatively long period of time, for example six weeks. The arm is therefore normally immobilised by means of an abduction pad or an abduction orthesis in an abduction position between 30° to 60° (depending on the tone of the refixed tendons determined intraoperatively). In order to keep the above problems caused by insufficient movement to a minimum, exercises are carried out by passive movements of the shoulder, for which the help of an assistant is necessary, for example a physiotherapist or a suitable trained employee. The aftercare may also be supported by the use of a CPM (continuous passive motion) chair. In this case, the arm is laid on a positioning rest of a special treatment chair and moved passively in the shoulder joint in a definable range by means of motor force. However, this method is very cost intensive and involved.

However, owing to the time restriction, the passive movement of a shoulder joint with the aid of a therapist is not sufficiently suitable to replace the movements carried out spontaneously during daily use and to reliably prevent immobilisation damage.

A dynamic shoulder joint orthesis in the form of a shoulder abduction orthesis according to the preamble of claim 1 is further known from DE 84 07 242 U1. This orthesis makes it possible to move the upper arm splint against the resistance of a spring in the adduction or abduction direction over a specific angular range. For this purpose the splint comprises a guide means which can be fastened on the upper body and comprises a housing, in which the lower end of a support rod is displaceably guided.

With the aid of ortheses of this type it is possible to adjust the orthesis by selecting a suitable spring, in such a way that it holds the upper arm in a specific abduction position or guides it back into this position without the patient having to engage the corresponding muscles.

In the shoulder abduction orthesis known from DE 84 07 242 U1, the upper arm splint consists of a proximal upper arm splint part connected in an articulated manner to the guide means and a distal upper arm splint part which is connected rigidly to the proximal upper arm splint part. The upper arm laid in the shell-shaped upper arm support is fixed in the upper arm support by means of a fastening strip guided over the upper arm.

However, it has been found that the rigid fixing of the upper arm associated with this is not optimal, since in the fixing position the upper arm does not move in a circular path about the orthesis hinge arranged beneath the shoulder joint during an adduction or abduction movement, thus causing relative movements between the upper arm and upper arm support.

The object of the invention is to provide a shoulder joint orthesis of the type mentioned at the outset, with which a physiologically improved guidance of the upper arm is made possible during adduction and abduction movements.

This object is achieved in accordance with the invention by a shoulder joint orthesis having the features of claim 1. Advantageous embodiments of the invention are described in the further claims.

In the shoulder joint orthesis according to the invention the distal upper arm splint part is floatingly mounted on the proximal upper arm splint part such that the distance between the upper arm support and the hinge can be changed during a movement in the adduction and abduction directions.

Owing to the freely displaceable mounting of the distal upper arm splint part on the proximal upper arm splint part, the distance between the upper arm support and the orthesis hinge is adapted, during each phase of the adduction and abduction movements, to the distance between the upper arm and the shoulder joint in the fixing position. Shear and compressive forces in the longitudinal direction of the upper arm and relative movements of the upper arm in relation to the upper arm support can thus be excluded. The shoulder joint orthesis according to the invention thus makes it possible to guide the upper arm in a physiologically improved manner during adduction and abduction movements.

In accordance with an advantageous embodiment of the invention, the distal upper arm splint part comprises a splint housing into which the proximal upper arm splint part protrudes, wherein the splint housing surrounds the proximal upper arm splint part at least in part and is mounted displaceably on the proximal upper arm splint part in a telescopic manner. Alternatively, it would however also be possible to provide the splint housing on the proximal upper arm splint part and to guide the distal upper arm splint part displaceably within the proximal splint housing.

The distal upper arm splint part is advantageously guided by means of a ball bearing on the proximal upper arm splint part. As a result, a very smooth, precise guidance of the distal upper arm splint part is ensured. Alternatively, however, a sliding bearing is also conceivable between the proximal and distal upper arm splint parts.

A very stable, precise and smooth sliding guidance is provided if the ball bearing comprises first bearing rails which are fixed on the proximal upper arm splint part, and second bearing rails which are fixed on the distal upper arm splint part and overlap, in part, the first bearing rails, wherein the first and second bearing rails are each U-shaped in cross-section and comprise raceways arranged in their side branches for receiving balls.

The splint housing of the distal upper arm splint part is advantageously U-shaped in cross-section, wherein the splint housing overlaps the proximal upper arm splint part on the upper face thereof. Such a splint housing affords a specific level of protection to the bearing and constitutes an aesthetically pleasing casing for the bearing parts.

According to an advantageous embodiment, the distal upper arm splint part is guided in a longitudinally displaceable manner on the proximal upper arm splint part by means of a roller bearing, the roller bearing comprising at least four first rollers which are rotatable about first rotation axes which are parallel to each other, and at least four second rollers which are rotatable about second rotation axes parallel to each other and arranged perpendicular to the first rotation axes. In this case the rollers are expediently mounted on the proximal upper arm splint part, the splint housing of the distal upper arm splint part having running surfaces in its side regions, on which running surfaces the rollers rest. This embodiment has the advantage that bearing rails which protrude above the distal splint housing are not necessary. Furthermore, this bearing is very smooth-running, stable and dirt-resistant.

The invention will now be described in greater detail and by way of example on the basis of the drawings, in which:

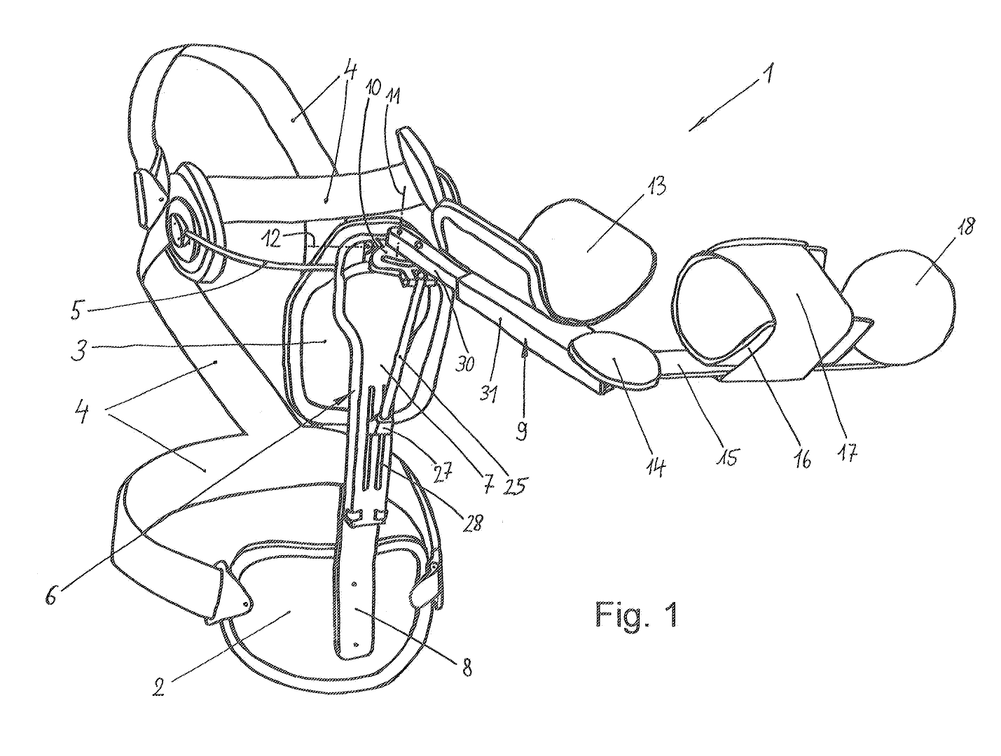

FIG. 1: is a three-dimensional view of a shoulder joint orthesis according to the invention;

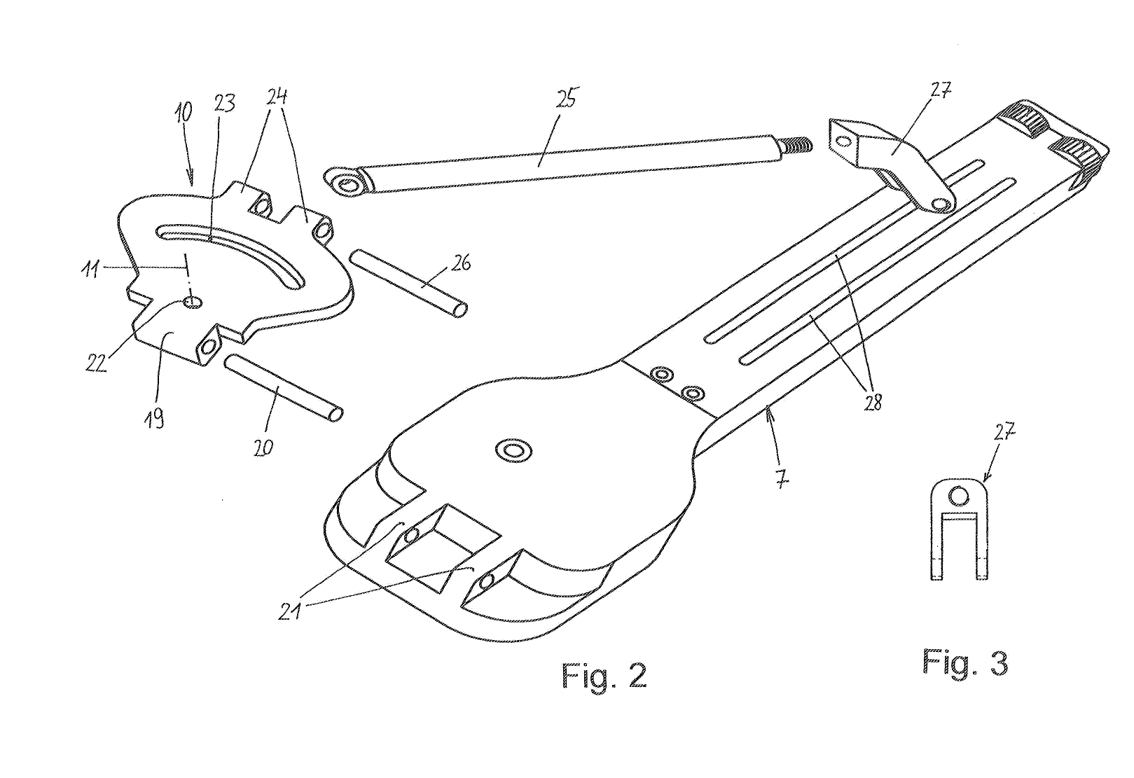

FIG. 2: is an exploded view of the guide means, support rod, hinge plate and the support rod coupling part;

FIG. 3: is a front view of the coupling part of FIG. 2;

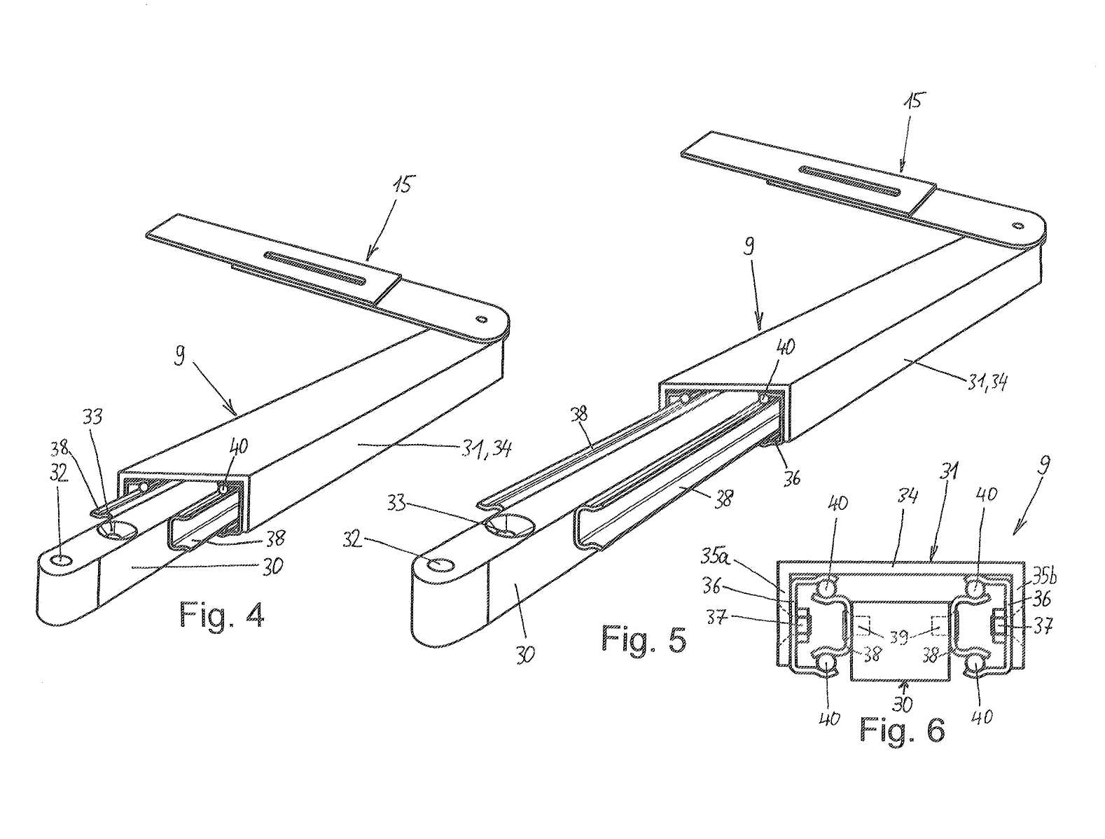

FIG. 4: is a three-dimensional view of a first embodiment of the proximal and distal upper arm splint parts in the largely inserted state, with lower arm splint;

FIG. 5: is a view according to FIG. 4, wherein the proximal and distal upper arm splint parts are pulled apart further;

FIG. 6: is an end view of the upper arm splint according to the first embodiment;

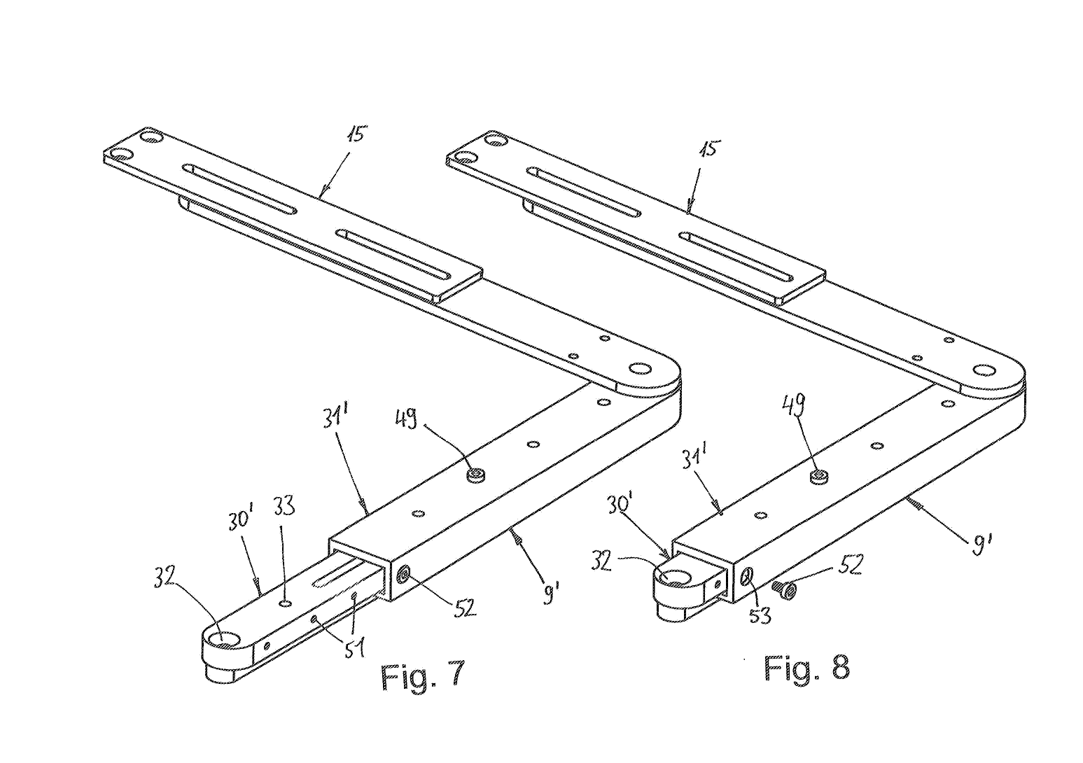

FIG. 7: is a three-dimensional view of a second embodiment of the proximal and distal upper arm splint parts in the largely pulled apart state, with lower arm splint;

FIG. 8: is a view according to FIG. 7, wherein the proximal and distal upper arm splint parts are largely pushed together;

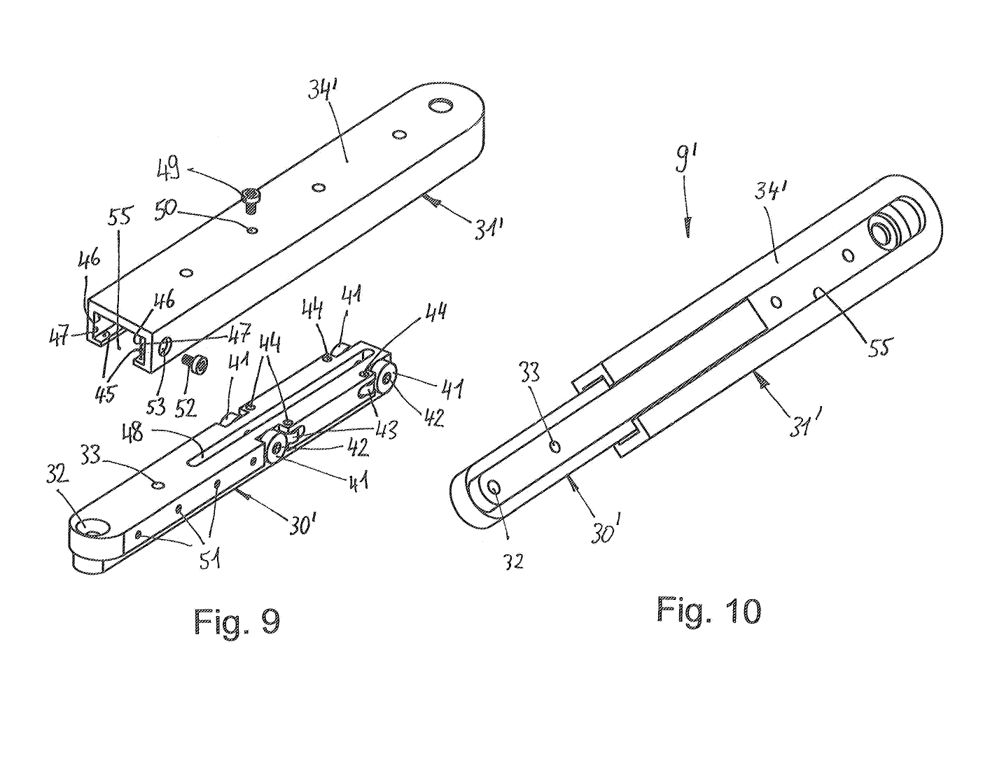

FIG. 9: is a separate view of the proximal and distal upper arm splint parts of the second embodiment; and

FIG. 10: shows the upper arm splint parts of the second embodiment shown at an angle from below.

The shoulder joint orthesis 1 illustrated in FIG. 1 comprises a lower support element 2, which rests laterally against the upper body in the hip region, and an upper contact element 3, which rests laterally against the chest directly beneath the shoulder. The support element 2 and contact element 3 are fastened to the upper body by means of a harness which, in the embodiment illustrated, comprises a belt 4 and a stable steel clasp 5.

The support element 2 and contact element 3 fix a stable guide means 6 which comprises an elongate housing 7 and an extension bar 8 fixed thereto. The housing 7 and extension bar 8 expediently consist of metal, for example aluminium. In the assembled state of the shoulder joint orthesis 1, the longitudinal axis of the housing 7 and of the extension bar 8 extend substantially vertically, wherein the housing 7 is fastened to the upper contact element 3 and extends as far as the vicinity of the shoulder, that is to say as far as the vicinity of the patient's armpit area, whereas the longitudinal bar 8 is fastened to the lower end of the housing 7 and to the lower support element 2 and, depending on length, determines the distance between the support element 2 in the vicinity of the hip and the upper contact element 3.

In order to hold the arm of a patient in the abducted position and/or to guide it in a supported manner in the abduction and adduction directions, an upper arm splint 9 is mounted in an articulated manner in the upper end region of the housing 7 by means of a hinge plate 10. In order to adjust the length of the upper arm splint 9, said splint consists of two upper arm splint parts 30, 31 which are guided inside one another in a telescope-like manner and can be displaced relative to one another so as to vary the extension length. The upper arm splint 9 is also pivotable at one end relative to the hinge plate 10 about a pivot axis 11, so that the upper arm splint 9 can be fixed to the hinge plate 10 at different angles. A circular arc-shaped slot 23 is provided in the hinge plate 10 for this purpose, through which slot a screw (not shown) can be guided, with which the proximal upper arm splint part 30 is fixed to the hinge plate 10. The pivot axis 11 extends perpendicular to the pivot axis 12 about which the hinge plate 10 can be pivoted relative to the housing 7 in the abduction and adduction directions.

A half-shell-shaped upper arm support 13 is fixed to the upper face of the distal upper arm splint part 31, in which support the upper arm can be laid and also expediently fixed by means of a fastening strip (not shown). When the distal upper arm splint part 31 is displaced relative to the proximal upper arm splint part 30, the upper arm support 13 is thus accordingly entrained, so that the distance between the upper arm support 13 and the orthesis articulation can be changed.

The upper arm splint 9 is connected at its distal end via an articulation arranged beneath a pad 14 to a lower arm splint 15. A half-shell-shaped lower arm support 16 is fixed in said lower arm splint, in which support the lower arm can be laid. The lower arm can be fixed in the lower arm support 16 by means of a fastening strip 17. A hand support 18, which in particular may take the form of a round or spherical pad which enables the patient to carry out kneading exercise with his fingers, is located at the distal end of the lower arm splint 15.

The hinge plate 10 forms a living hinge with the guide means 6 and for this purpose comprises at one end a hinge tab 19 (FIG. 2) which is connected in an articulated manner to bearing webs 21 of the housing 7 via a hinge pin 20. In order to mount the upper arm splint 9 so as to be pivotable relative to the hinge plate 10 about the pivoting axis 11, a hole 22 is provided in the vicinity of the hinge tab 19, into which hole a hinge pin (not shown) is introduced.

The upper arm splint 9 is fixed in the proximal end region of the upper arm splint part 30 to the hinge plate 10. A corresponding pivoting of the hinge plate 10 in the adduction and abduction directions is thus coupled with a corresponding pivoting of the upper arm splint 9 and thus also of the lower arm splint 15.

The hinge plate 10 further comprises, at its end opposite the hinge tab 19, two hinge tabs 24 which connect the upper end of a support rod 25 in an articulated manner. For this purpose, the support rod 25 is introduced via its upper end between the hinge tabs 24 and fixed by means of a hinge pin 26.

The upper arm splint 9 and therefore the patient's arm lying thereon is held in the desired abduction positions relative to the upper body by means of support rods 25 or exerts a support force from beneath onto the hinge plate 10 and therefore onto the upper arm splint 9 during corresponding abduction and adduction movements. The lower end of the support rod 25 engages via a prong-shaped coupling element 25 in two parallel longitudinal slits 28 in the housing 7 and can be displaced along the longitudinal slit 28 either against the action or with the assistance of a spring mechanism arranged in the housing 7. The spring force can be net in such a way that the support force acting on the upper arm splint 9 compensates for the weight of the patient's arm in any abduction position of the arm such that the arm can be moved weightlessly, that is to say without any nominal active muscular support, in the abduction direction. Furthermore, it is also possible to set the spring force so that the shifting force applied by the spring mechanism and acting on the support rod 25 is greater than the weight of the arm including the arm splint so that an active passive mobilisation treatment of the shoulder joint is enabled in the abduction direction. Further, it is possible to fix the support rod 25 in any desired position of the range of displacement, that is to say a continuous fixing of the upper arm splint 9 and therefore a static fixing of the arm in any abduction position is possible.

The structure and operating principle of a first embodiment of the upper arm splint 9 according to the invention will be described hereinafter in greater detail with reference to FIGS. 4 to 6.

As already mentioned, the upper arm splint 9 comprises a proximal upper arm splint part 30 and a distal upper arm splint part 31, which is mounted on the proximal upper arm splint part 30 so as to be longitudinally displaceable.

The proximal upper arm splint part 30 comprises at its proximal end a bore 32 for receiving a hinge pin (not shown) which projects into the bore 22 in the hinge plate 10 so as to hold the upper arm splint 9 on the hinge plate 10 pivotably about the pivot axis 11. The angular position set can then be fixed by means of a screw (not shown) which is passed through a bore 33 and penetrates the circular arc-shaped slot 23 in the hinge plate 10.

Furthermore, the proximal upper arm splint part 30 consists of a profile element which is rectangular in cross-section, on which the distal upper arm splint part 31 is mounted so as to be longitudinally displaceable.

For this purpose the distal upper arm part 31 comprises a splint housing 34 which is U-shaped in cross-section and overlaps the proximal upper arm splint part 30 laterally on the upper face thereof. The shell-shaped upper arm support 13 is fixed on the upper face of the splint housing 34. Bearing rails 36 which are U-shaped in cross-section and extend longitudinally are fixed on the inner face of the side branches 35a, 35b by means of screws 37.

Bearing rails 38 which likewise extend longitudinally and are U-shaped in cross-section are fixed to the lateral outer faces of the proximal upper arm splint part 30 by means of screws 39. The inner, laterally outwardly open bearing rails 38 are smaller than the outer, laterally inwardly open bearing rails 36 and protrude into the adjacent outer bearing rails 36 such that the side branches of the bearing rails 36, 38 overlap in part. The bearing rails 36, 38 comprise raceways in this overlapping region for receiving balls 40, by which the outer bearing rails 36 and therefore the distal upper arm splint part 31 are guided on the inner bearing rails 38 of the proximal upper arm splint part 30.

Together with the balls 40, the bearing rails 36, 38 form a smooth, stable and precise ball bearing with which the distal upper arm splint part 31 can be displaced relative to the proximal upper arm splint part 30 in a telescopic manner.

Since, as can be seen in FIG. 1, the upper arm support 13 is fixed on the distal upper arm splint part 31, the distance between the upper arm support 13 and the pivot axis 12 can change during an adduction and abduction movement of the upper arm and can be continuously adapted optimally to the curve of movement of the upper arm.

A second embodiment of an upper arm splint 9′ according to the invention is described hereinafter with reference to FIGS. 7 to 10. This upper arm splint differs from the upper arm splint 9 which has been described with reference to FIGS. 1 to 6 in terms of the type of bearing with which the distal upper arm splint part 31′ is floatingly mounted on the proximal upper arm splint part 30′. The remaining parts of the shoulder joint orthesis, which have been described with reference to FIG. 1 in particular, are, in contrast, also present unmodified in the second embodiment and are therefore not described in further detail.

As can be seen in FIGS. 7 to 10, the upper arm splint 9′ comprises a proximal upper arm splint part 30′ and a distal upper arm splint part 31′, which is mounted on the proximal upper arm splint part 30′ so as to be longitudinally displaceable. The distal upper arm splint part 31′ is floatingly mounted on the proximal upper arm splint part 30′ by means of a roller bearing, so as to be longitudinally displaceable. By raising and lowering the upper arm, the length of the upper arm splint 9′ is therefore automatically adjusted to the distance between the upper arm support 13 and the shoulder joint of the patient, which distance varies when the upper arm is pivoted.

The roller bearing comprises two pairs of first rollers 41 which are mounted on the proximal upper arm splint part 30′, so as to be rotatable about two parallel first rotation axes 42. A pair of the first rollers 41 is arranged in the distal end portion of the proximal upper arm splint part 30′, while the second pair is arranged in a central portion. Furthermore, the first rollers 41 are situated in the two side regions of the proximal upper arm splint part 30′ and protrude upwards and downwards slightly beyond the outer contour of the upper arm splint part 30′.

Furthermore, the roller bearing comprises four second rollers 43 which are likewise arranged on the proximal upper arm splint part 30′ and in proximity to the first rollers 41. These second rollers 42 are mounted so as to be rotatable about four second rotation axes 44 which extend parallel to each other and perpendicular to the first rotation axes 42. The second rollers 43 protrude slightly beyond the side surfaces on the two sides of the proximal upper arm splint part 30′.

In the mounted state of the distal upper arm splint part 31′, the first rollers 41 roll on lower guide rails (running surfaces) 45 or upper guide rails 46 of the distal upper arm splint part 31, depending on whether this is loaded from above or below. The second rollers 43 roll on lateral guide rails 47 of the distal upper arm splint part 31′, whereby this is guided laterally.

The guide rails 45, 46, 47 are expediently formed by the inner faces of the splint housing 34′ of the distal upper arm splint 31′ such that no separate parts are necessary for this purpose.

As can be seen in particular in FIG. 9, a central longitudinal groove 48 is further provided on the upper side of the proximal upper arm splint part 30′. A screw 49 which can be screwed into a corresponding tapped hole 50 in the distal upper arm splint part 31′ protrudes into this longitudinal groove. This screw 49 serves as an extension limit stop for the distal upper arm splint part 31′, in that the portion of the screw 49 which protrudes into the longitudinal groove 48 strikes against the distal end of the longitudinal groove 48 when the distal upper arm splint part 31 reaches the maximum allowable extension length.

A plurality of tapped holes 51 which are spaced apart from each other are further provided in a side wall of the proximal upper arm splint part 30′, into which holes a locking screw 52 can be screwed when the distal upper arm splint part 31′ is to be fixed in certain positions of extension on the proximal upper arm splint part 30′. For this purpose, the locking screw 52 is fed through a side hole 53 in the distal upper arm splint part 31′.

In the embodiment shown in FIGS. 7 to 10, the splint housing 34′ of the distal upper arm splint part 31′ has a central longitudinal slot 55 in the lower housing wall. Alternatively, however, it is also perfectly possible to form the lower housing wail in a closed manner, resulting in a peripherally closed cross-section for the splint housing 34′.

1. Dynamic shoulder joint orthesis, in particular a shoulder abduction orthesis, comprising:
a guide means which can be fastened on the upper body,
an upper arm splint which is fastened by means of a hinge to the guide means, can be moved at least in the adduction and abduction directions, and comprises a proximal upper arm splint part and a distal upper arm splint part fixed thereto, which carries an upper arm support, a support rod for supporting the upper arm splint, the support rod comprising a lower end which is guided displaceably on the guide means, a spring for applying a displacement force to the lower end of the support rod.
characterised in that the distal upper arm splint part is mounted floatingly in the longitudinal direction on the proximal upper arm splint part so that the distance between the upper arm support and the hinge can be changed during a movement in the adduction and abduction directions.

2. Shoulder joint orthesis according to claim 1, characterised in that the distal upper arm splint part comprises a splint housing into which the proximal upper arm splint part protrudes, the splint housing surrounding the proximal upper arm splint part, at least in part, and being displaceably mounted on the proximal upper arm splint part in a telescopic manner.

3. Shoulder joint orthesis according to claim 1, characterised in that the distal upper arm splint part is guided in a longitudinally displaceable manner on the proximal upper arm splint part by means of a ball bearing.

4. Shoulder joint orthesis according to claim 3, characterised in that the ball bearing comprises first bearing rails which are fixed on the proximal upper arm splint part, and second bearing rails which are fixed on the distal upper arm splint part and overlap the first bearing rails in part, the first and second bearing rails each having a U-shaped cross-section and comprising raceways arranged in their side branches for receiving balls.

5. Shoulder joint orthesis according to claim 2, characterised, characterised in that the splint housing of the distal upper arm splint part is U-shaped in cross-section and overlaps the proximal upper arm splint part on the upper face thereof.

6. Shoulder joint orthesis according to claim 1, characterised in that the distal upper arm splint part is guided in a longitudinally displaceable manner on the proximal upper arm splint part by means of a roller bearing, the roller bearing comprising at least four first rollers which are rotatable about first rotation axes which are parallel to each other, and at least four second rollers which are rotatable about second rotation axes which are parallel to each other and arranged perpendicular to the first rotation axes.
